(19) United States
(12) Patent Application Publication
(10) Pub. No.: US 2013/0072903 A1
(43) Pub. Date: Mar. 21, 2013

(54) Adipose Tissue Graft for Wound Healing

(71) Applicant: John Chapman, Sacramento, CA (US)

(72) Inventor: John Chapman, Sacramento, CA (US)

(21) Appl. No.: 13/623,576

(22) Filed: Sep. 20, 2012

Related U.S. Application Data
(60) Provisional application No. 61/536,997, filed on Sep. 20, 2011.

(51) Int. Cl. A61M 37/00

(52) U.S. Cl. 604/522

(57) ABSTRACT

An improved method for preparing an adipose tissue biocomposite graft to serve a wide range of medical applications is presented. In particular, the embodiments consider a performing lipoplasty to derive a plurality of adipose tissue fragments containing at least one viable stem cell from a donor, harvesting said plurality of adipose tissue fragments from said donor, placing said plurality of adipose tissue fragments in contact with appropriate concentrations of a thrombin source and a fibrinogen source to achieve an appropriate gelling reaction and applying the mixture of said adipose tissue fragments, said thrombin source and said fibrinogen source to a wound site of the donor so as to promote wound healing. The adipose biocomposite graft of the present invention can be easily processed, molded and customized to precise dimensions. The present invention employs a three dimensional mold cavity to prepare multiple castings of said adipose tissue biocomposite grafts for an individual donor.

### RELATED APPLICATIONS

This application is a Nonprovisional Application of and claims priority to U.S. Provisional Patent Application 61/536997, filed Sep. 20, 2011. This patent application is incorporated herein in its entirety as if set out in full.

### BACKGROUND OF THE DISCLOSURE

1. Technical Field of the Disclosure

The present embodiment relates in general to methods for the treatment of tissue injuries or defects. Specifically, the present invention relates to an adipose tissue based wound sealant and a method for its delivery in a three dimensional matrix to a wound site to promote wound healing including reducing fluid extravasation and reducing scarring.

2. Description of the Related Art

The repair of damaged tissues is of universal concern to all surgical specialties. Damage to tissue, often as the result of the surgical procedure, can be difficult to repair. Continued fluid extravasation, drying of tissue, infection, can result in increased patient morbidity, prolonged recovery, scarring and the defeat of an otherwise promising outcome.

Wound healing is a complex and dynamic process. Once a wound begins healing, normally the process resolves with complete wound closure. However, healing of acute and chronic wounds can become impaired by patient factors such as diabetes and/or wound factors such as infection. Restarting a wound with impaired healing is difficult because good standard wound care does not always provide an improved healing outcome and more advanced tissue grafting may be required.

Skin grafting is primarily intended for tissue reconstruction. The skin grafting method involves the procurement of living cells from a body; and returning the skin graft containing the living cells to the donor within the same surgical procedure. Skin grafting can improve patient outcomes in the case of tissue damage due to surgical injury, traumatic injury, structural defects, or reconstructive surgery. These injuries typically include the loss of adipose tissue. Attempts to engineer adipose tissue have led to different harvesting and preparation techniques to increase adipose tissue viability.

Fat grafting involves the harvest of adipose tissue from one location and re-implantation in another location. Fat grafts have been utilized for soft-tissue augmentation for more than one hundred years in a diverse range of reconstructive and aesthetic procedures. Autologous fat transfer is commonly used to achieve cosmetic effects. The advent of liposuction techniques, abundant donor-tissue availability, and the relative ease of harvesting has made autologous fat an attractive material for use as soft-tissue filler. While performing autologous fat transfer, the fat is aspirated from the subcutaneous layer, usually the abdominal wall by means of a suction syringe, and injected into the subcutaneous tissues overlying the depression. Common harvesting techniques include syringe aspiration and vacuum pump aspiration. In practicing such procedures, only some of the injected fat survives and consequently the amount of fat injected is strategically in excess of that needed for filling the depression.

Recent advancements in the art provide a method for augmenting autologous fat transfer. The method includes removing adipose tissue from a patient, processing a portion of the adipose tissue to obtain a substantially isolated population of regenerative cells; mixing the regenerative cells with another portion of adipose tissue to form a composition; and administering the composition to the patient from whom the adipose tissue was removed. The composition may be implanted into the recipient to provide autologous soft tissue filler for correction of contour defects such as wrinkles, divots, pockmarks, and larger deficits, or for providing support to damaged structures such as the urethra. The composition may also be administered to breast regions in connection with breast augmentation procedures and soft tissue defects. However, the fat grafts do not act as a wound sealant due to their being simple tissue fragment suspensions.

One of the existing systems provides a method for making enhanced, autologous fat grafts. The method includes removal of adipose tissue from a patient using a tissue removal system and processing at least a part of the adipose tissue to obtain a concentration of stem cells. The processing of adipose tissue results in more concentration of stem cells of the adipose tissue than before processing and the stem cells are administered to a patient. This method is practiced in a closed system so that the stem cells are not exposed to an external environment prior to being administered to a patient. The adipose tissue is separated from non-adipose tissue using a tissue collection container that utilizes decantation, sedimentation, and/or centrifugation techniques to separate the materials. The main drawback of this system is its higher cost, time required for additional preparation steps, and increased complexity in that cells are subjected to enzymatic disruption with its inherent cost. Further this material is difficult to control and does not act as a wound sealant due to it being an amorphous, simple tissue suspension.

In U.S. Patent Application No. US 2006/0134781 A1 filed Dec. 7, 2005 entitled “Three-Dimensional Cell Culture System” disclosed by Young Il Yang and Nancy Jane Shelby, describe a three-dimensional culture system so as to provide an efficient mechanism for in vitro production of stem cells derived from adipose tissue. The authors disclose in the patent application and in subsequent publications (Journal of Cell Physiology 2010 224 (3):807-16 and Acta Biomater. 2011 7 (12):4109-19), the entire contents are incorporated herein by reference, a three-dimensional matrix to incorporate the adipose tissue to incorporate adipose tissue into the three-dimensional matrix. After incubation, the 3-D matrix may be degraded to liberate stem cells, or progeny cells arising from the 3-D matrix. The disclosure provides a means to expand stem cells from adipose tissue fragments in vitro but does not address the problems of improving clinical wound healing.

Another existing system described in U.S. Pat. No. 8,137,702, a conformable tissue implant for use in treating injured soft tissue, and a method for delivering such an implant in a minimally invasive procedure. The tissue repair implant comprises a tissue carrier matrix comprising a plurality of biocompatible, bioresorbable granules and at least one tissue fragment in association with the granules. The tissue fragment contains one or more viable cells that can migrate from the tissue and populate the tissue carrier matrix. The tissue fragments serve as a cell source for new cellular growth, and have an effective amount of viable cells that can migrate out of the tissue fragment and populate the tissue carrier matrix once the implant is delivered to the patient. The granules serve as a microcarrier to provide sufficient mechanical integrity for cellular integration with the surrounding environment during the tissue remodeling process. The tissue carrier matrix can be provided with a binding agent that enables the implant to form a gel-like or semi-solid implant. A curing agent can additionally be provided to enable the implant to set either before or after delivery to the implantation site. The finely minced tissue fragments and granules together form an injectable solution that can be delivered by injection in a minimally invasive procedure. This method allows for delivering the tissue implant that is able to conform to any defect size, shape, or geometry of the implantation site. A downside of this method is the higher cost, additional preparation steps, the risk of exposing the body to exogenous biomaterial granules and overall increased complexity.

Fibrin sealants are prepared by applying a composition containing a sufficient amount of thrombin, such as human, bovine, ovine or porcine thrombin, to the site to a sufficient concentration of fibrinogen to be converted to the fibrin, which then solidifies in the form of a gel. The inclusion of living cells in a fibrin sealant has been performed using stem cells to treat wounds and blood cells including platelets to form platelet gels. Platelet rich plasma (PRP) gel is considered to be advanced wound therapy for chronic and acute wounds. For more than 20 years, PRP gel has been used to stimulate wound healing. Autologous PRP gel consists of cytokines, growth factors, chemokines, and a fibrin scaffold derived from a patient's blood. The mechanism of action for PRP gel is thought to be the molecular and cellular induction of normal wound healing responses similar to that seen with platelet activation. Strong evidence for efficacy of PRP gel in the clinical setting is, however, lacking.

It can therefore be seen that known methods and techniques for enhancing wound healing fall short of providing a reliable, biocompatible, therapeutically effective composition readily prepared at the point of care using the patient's own tissues to enhance wound healing. Exogenous materials induce inflammatory foreign body compositions which delay or inhibit healing. Homologous products carry risk of pathogen transmission or biocompatibility reactions. Accordingly, there remains a need for an effective wound sealant which is safe, easily and quickly prepared using the patient's own autologous cells, readily scalable to treat small or large wounds, and which augments the healing process and preferably reduces the frequency of scarring.

Based on the foregoing there is a demonstrable need for the preparation of an adipose tissue biocomposite graft by harvesting healthy living tissue from a donor; processing the tissue to form the adipose tissue biocomposite graft; and transplanting the adipose tissue biocomposite graft to the donor at the site of an injury, wound or structural defect to enhance healing within a single surgical procedure. The present invention will result in the production of an adipose tissue biocomposite graft that will allow cells within the graft to exit the tissue fragments as well as surrounding body cells to migrate into the graft and produce new tissue in patient's body. This process also provides the safest and most cost-effective, autologous graft system currently available designed for enhanced wound healing. The autologous tissue biocomposite grafts can be made in a quick and efficient manner for immediate use during the same surgery. This unique method overcomes prior art shortcomings by accomplishing these critical objectives.

### SUMMARY OF THE DISCLOSURE

To minimize the limitations found in the prior art, and to minimize other limitations that will be apparent upon the reading of the specifications, the preferred embodiment of the present invention provides an improved method for preparing and applying an adipose tissue biocomposite graft to a wound site for the medical purposes of reducing fluid extravasation, enhance wound healing and reduce scarring.

The present invention discloses performing lipoplasty to derive a plurality of adipose tissue fragments containing at least one viable stem cell from a donor, harvesting said plurality of adipose tissue fragments from said donor, placing said plurality of adipose tissue fragments in contact with appropriate concentrations of a thrombin source and a fibrinogen source to achieve an appropriate gelling reaction and applying the mixture of said adipose tissue fragments, said thrombin source and said fibrinogen source to a wound site of the donor so as to promote wound healing.

The above steps may occur within the same surgical procedure at the point of care, and said adipose tissue biocomposite graft may be applied to the donor in the form of a liquid biocomposite, a molded gel biocomposite and gel biocomposite fragments. The liquid biocomposite is prepared by mixing said adipose tissue fragments, said thrombin source and said fibrinogen source in liquid form to fill the wound site in the donor's body. The mixture of said adipose tissue fragments, said thrombin source and said fibrinogen source may be injected into a three dimensional mold cavity so that the adipose tissue biocomposite graft forms into a three dimensional shaped gel biocomposite. In each case, the structure of said adipose tissue biocomposite graft is achieved by controlling the relative percentage of the graft volume derived from adipose tissue fragments and controlling concentrations of said fibrinogen source and said thrombin source. The adipose tissue fragments constitute 10% to 90% of the total volume of said adipose tissue biocomposite graft.

During the processing of the adipose biocomposite graft, it is important to provide the graft with supplements to enhance the therapeutic potential of the material. These supplements should be added to the mixture prior to the initiation of the gelling reaction. By supplementing the graft with such materials, customized and more therapeutic grafts can be prepared.

In accordance with another aspect of the present invention, a method for preparing an improved adipose tissue biocomposite graft with a wound-healing promoter to serve a wide range of medical applications is disclosed.

In accordance with yet another aspect of the present invention, a method for preparing an improved adipose tissue biocomposite graft utilizing a syringe to serve a wide range of medical applications is presented.

A first objective of the present invention is to provide an adipose tissue biocomposite graft that may serve a wide range of medical applications.

A second objective of the present invention is to provide an adipose tissue biocomposite graft characterized by elasticity and high tensile strength, allowing it to be easily handled by an operator.

A third objective of the present invention is to provide a biocomposite graft, which could easily processed, molded, and customized to precise dimensions.

Another objective of the present invention is to provide a tissue biocomposite graft that can be supplemented with additives to obtain customized and more therapeutic grafts.

Yet another objective of the invention is to provide a method for generating fully autologous adipose tissue composite grafts from a donor to treat wounds in the safest and most cost-effective manner.

Still another objective of the invention is to provide a single three-dimensional mold to prepare multiple castings of the adipose tissue biocomposite grafts for an individual donor.

These and other advantages and features of the present invention are described with specificity so as to make the present invention understandable to one of ordinary skill in the art.

### BRIEF DESCRIPTION OF THE DRAWINGS

In order to enhance their clarity and improve understanding of these various elements and embodiments of the invention, elements in the figures have not necessarily been drawn to scale. Furthermore, elements that are known to be common and well understood to those in the industry are not depicted in order to provide a clear view of the various embodiments of the invention, thus the drawings are generalized in form in the interest of clarity and conciseness.

FIG. 1 is an operational flow chart of a preferred embodiment of a method for preparing an improved adipose tissue biocomposite graft to serve a wide range of medical applications in accordance with one aspect of the present invention;

FIG. 2 is an alternative operational flow chart of a method for preparing an improved adipose tissue biocomposite graft to serve a wide range of medical applications in accordance with another aspect of the present invention;

FIG. 3 is an alternative operational flow chart of a method for preparing an improved adipose tissue biocomposite graft with a wound-healing promoter to serve a wide range of medical applications in accordance with another aspect the present invention;

FIG. 4 is an alternative operational flow chart of a method for preparing an improved adipose tissue biocomposite graft utilizing a syringe to serve a wide range of medical applications in accordance with another aspect of the present invention;

FIG. 5 illustrates the adipose tissue biocomposite graft prepared using a rectangular three dimensional mold cavity in accordance with an exemplary embodiment of the present invention; and

FIG. 6 illustrates the adipose tissue biocomposite graft prepared using a circular three dimensional mold cavity in accordance with the exemplary embodiment of the present invention.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below. Finally, many of the steps are presented below in an order intended only as an exemplary embodiment. Unless logically required, no step should be assumed to be required earlier in the process than a later step simply because it is written first in this document.

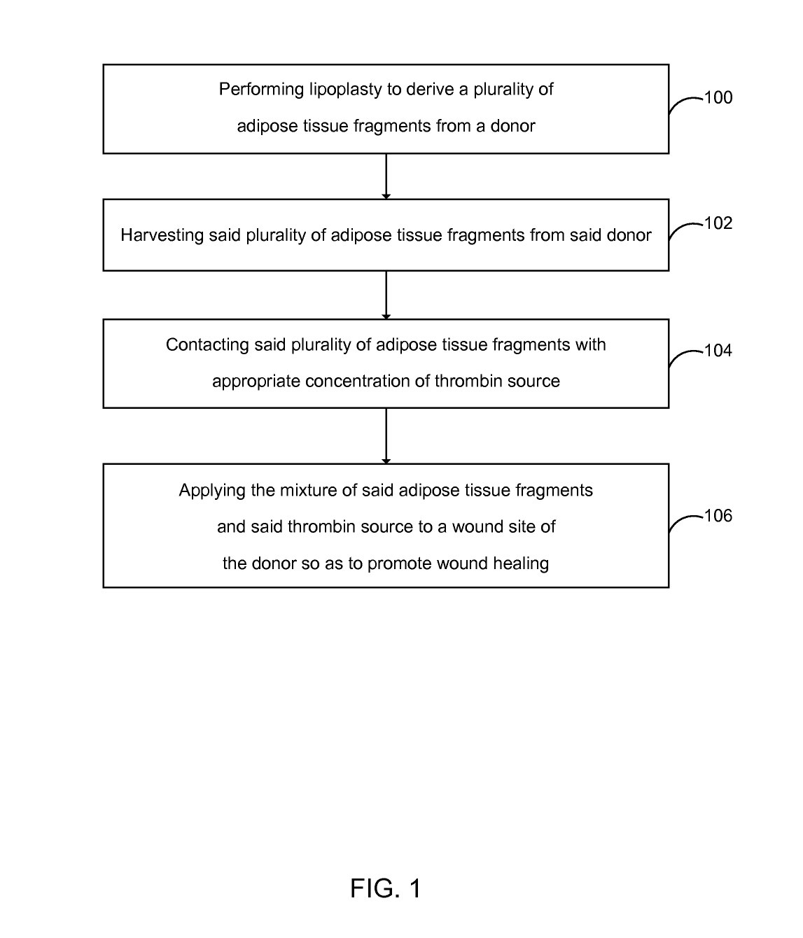

Preferred embodiment of the present invention considers a method for preparing an improved adipose tissue biocomposite graft to serve a wide range of medical applications. Referring to FIG. 1, an operational flow chart of the method preparing an improved adipose tissue biocomposite graft in accordance with one aspect of the present invention is illustrated. Initially, lipoplasty is performed to derive a plurality of adipose tissue fragments from a donor, as shown in block 100. Said plurality of adipose tissue fragments are harvested from said donor as indicated at block 102. Said plurality of adipose tissue fragments contains at least one viable stem cell. After harvesting said adipose tissue fragments, appropriate concentration of thrombin source is contacted with said adipose tissue fragments as shown in block 104. Finally, as indicated at block 106, the mixture of said adipose tissue fragments and said thrombin source is applied to a wound site of the donor so as to promote wound healing.

In this preferred embodiment, said adipose tissue fragments derive a fibrinogen source from the wound site of the donor. The promotion of wound healing includes at least one of the effects of: promoting hemostasis, reducing time for wound closure, reducing post-surgical wound complications and reducing scarring. The present invention focuses on a fully autologous graft system. In a particularly advantageous embodiment of the present invention, all the biological constituents comprising the biocomposite are autologous. The present invention teaches the method for preparing fully autologous biocomposite as explained below. The adipose tissue fragments thus derived by lipoplasty may have excess liquid which can be removed by draining, by removal of supernatant after gentle centrifugation, by filtration or after spontaneous phase separation due to density differences in the tissue fragments and suspending fluid as occurs with adipose tissue. The structure of said adipose tissue biocomposite graft thus prepared, is controlled by controlling the relative percentage of the graft volume derived from adipose tissue fragments and controlling concentrations of said thrombin source. Preferably, the concentration of thrombin source is 0.5 to 500 units/gram of said adipose tissue biocomposite graft. The adipose tissue fragments constitute 10% to 90% of the total volume of said adipose tissue biocomposite graft.

The most preferred tissue source for the practice of the present invention is the preparation of tissue fragments from autologous adipose tissue fragments contained in lipoaspirate. The adipose tissue fragments provide an abundant source of living cells for tissue engineering purposes and are safe for the donor so that even large amounts of adipose tissue can be removed from the body without significant untoward effect. The purpose of the adipose biocomposite graft is to be a biological volume replacement material that fills voids made at sites of injury and enhances wound healing. Biocomposites of adipose tissue fragments contain adipocytes, mesenchymal stem cells and endothelial precursor cells that can secrete growth factors important for angiogenesis particularly when exposed to thrombin. Revascularization of tissue through the process of angiogenesis is fundamental to wound healing. Therefore, the adipose tissue graft is intended to promote wound repair by reducing the risk of post-surgical complications such as delayed wound closure, scarring, fibrosis, wound re-opening, excessive wound inflammation and bacteria wound infection. The graft may be used in medical applications selected from a group consisting of: cosmetic, therapeutic and surgical procedures. In some other cases, the graft will provide a temporary support system or scaffolding that allows cells from the surrounding body tissues to migrate in and start producing new tissue.

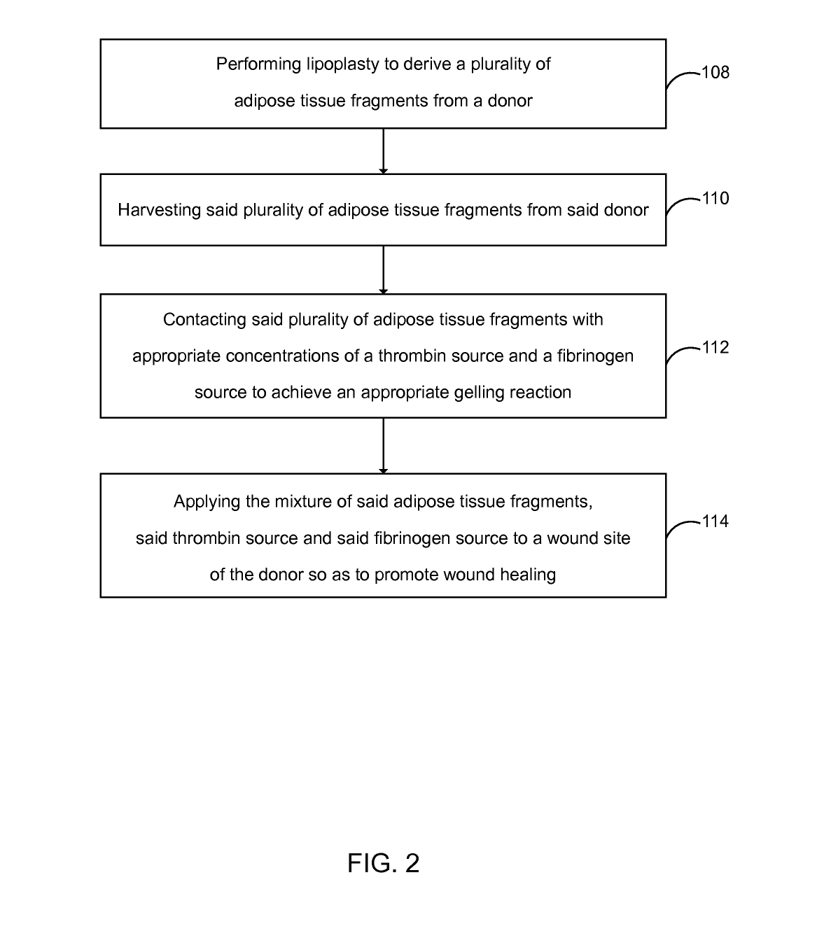

Turning now to FIG. 2, an alternative operational flow chart of a method for preparing an improved adipose tissue biocomposite graft to serve a wide range of medical applications in accordance with another aspect of the preferred embodiment of the present invention is illustrated. Initially, lipoplasty is performed to derive a plurality of adipose tissue fragments from a donor, as shown in block 108. Said plurality of adipose tissue fragments are harvested from said donor as indicated at block 110. In next step, as shown in block 112, said plurality of adipose tissue fragments are contacted with appropriate concentrations of a thrombin source and a fibrinogen source to achieve an appropriate gelling reaction. Finally, the mixture of said adipose tissue fragments, said thrombin source and said fibrinogen source are applied to a wound site of the donor so as to promote wound healing as indicated at block 114.

The mixture of said adipose tissue fragments, said thrombin source and said fibrinogen source is applied to the wound site as shown in block 114, in the form of: a liquid biocomposite, a molded gel biocomposite and gel biocomposite fragments. The liquid biocomposite is prepared by mixing said adipose tissue fragments, said thrombin source, and said fibrinogen source in liquid form to fill said wound site in the donor's body. The biocomposite graft can also be applied in the form of said molded gel biocomposite, which is prepared by (a) injecting the mixture of said adipose tissue fragments, said thrombin source and said fibrinogen source into a three dimensional mold cavity to achieve the gelling reaction, (b) removing said adipose tissue biocomposite graft from said three dimensional mold cavity and (c) applying said adipose tissue biocomposite graft to the wound site of the donor so as to promote wound healing.

In the preferred embodiment of the present invention, the gelling reaction may be achieved by placing a fluid containing thrombin in contact with a fluid containing fibrinogen with said fluids acting to be a tissue fragment suspending fluid. As explained in block 112, said adipose tissue fragments are contacted with selected concentrations of thrombin and fibrinogen in to achieve an appropriate gelling reaction. The concentration of said thrombin and said fibrinogen is kept optimum to achieve the intended gelling reaction. The amount of thrombin should be selected to provide sufficient time for transferring the reaction mixture into the mold before the gelling reaction occurs but the amount should not be so low as to take too long for the gelling reaction to cure within a reasonable time. It is desirable to have a time period of at least 15 seconds to load the mold or deliver to the body of the donor. The preferred time required for the gelling reaction to occur is less than 10 minutes and most preferably in less than 3 minutes.

For the embodiment of molding the adipose biocomposite or applying the biocomposite as a liquid to the body, the most preferred amount of thrombin is that which causes a clot time of 15 to 30 seconds of human plasma at room temperature when added in equal volumes. The amount of thrombin selected for contact with the fibrinogen should provide the operator sufficient time to transfer the mixture to the wound site where the gelling reaction is desired to occur (e.g., in the range of 1-20 units of thrombin/mL). This concentration of thrombin provides adequate time for the operator to dispense the mixture to the mold or body site prior to the gelling reaction occurring. If too much thrombin is added, the gelling reaction will take place before the mixture is added to the mold or delivered to the body. Because the gelling reaction is an irreversible process and the nascent gels can readily be disrupted if disturbed during the curing process, it is quite important to avoid this excessive speed of gelling by ensuring there is not too much thrombin in the mixture. The concentration of thrombin should be sufficiently high that a relatively small volume compared to the graft volume is required to achieve the intended gelling reaction. The concentration of fibrinogen determines in positive fashion the overall tensile strength of the biocomposite. A higher concentration of fibrinogen may be employed to prepare a stronger more persistent gel biocomposite. A preferred concentration of fibrinogen is less than 15 mg/mL. Preferably, in this method, said concentration of thrombin source is 0.5 to 500 units/gram of said adipose tissue biocomposite graft and said concentration of fibrinogen source is 0.1 to 60 mg/gram of said adipose tissue biocomposite graft.

For the present invention, said thrombin source is selected from a group consisting of: autologous thrombin serum, autologous thrombin serum supplemented with ethanol, allogeneic thrombin serum, allogeneic thrombin serum supplemented with ethanol, bovine thrombin, recombinant thrombin, and human thrombin derived from pooled plasma. The fibrinogen source is selected from a group consisting of: autologous whole blood anti-coagulated with a calcium-chelating agent, platelet rich plasma with its associated growth factors, autologous plasma, autologous platelet rich plasma, plasma and collagen mixture as represented by Vitagel by Orthovita; purified allogeneic fibrinogen as represented by Tisseel/Tissucol and Beriplast products or by Quixil® consisting of a cross-linked allogeneic fibrinogen-fibronectin multimers and other naturally occurring adhesive glycoproteins to promote adhesion to collagen. A more convenient source of fibrinogen for the practice of the current invention is autologous plasma. A particularly preferred source of fibrinogen is platelet rich plasma with its associated growth factors to further enhance the therapeutic potential of the tissue biocomposite graft. In one means for practicing the invention, the tissue fragments are first contacted with plasma. The ratio of volume of plasma used to rinse the graft should be sufficient that the remaining extra-cellular fluid in the tissue fragments does not significantly dilute the plasma. After exposing the tissue fragments to the plasma, the excess plasma may be removed by draining the grafting, introducing an absorbent material to wick away excess plasma from the graft, or gentle centrifugation of the graft followed by aspiration of the excess plasma. The graft suspended in plasma may then be contacted with a thrombin source. The tissue fragments can be mixed with plasma utilizing two syringes connected by a female-to-female luer lock connector or in-line static mixers.

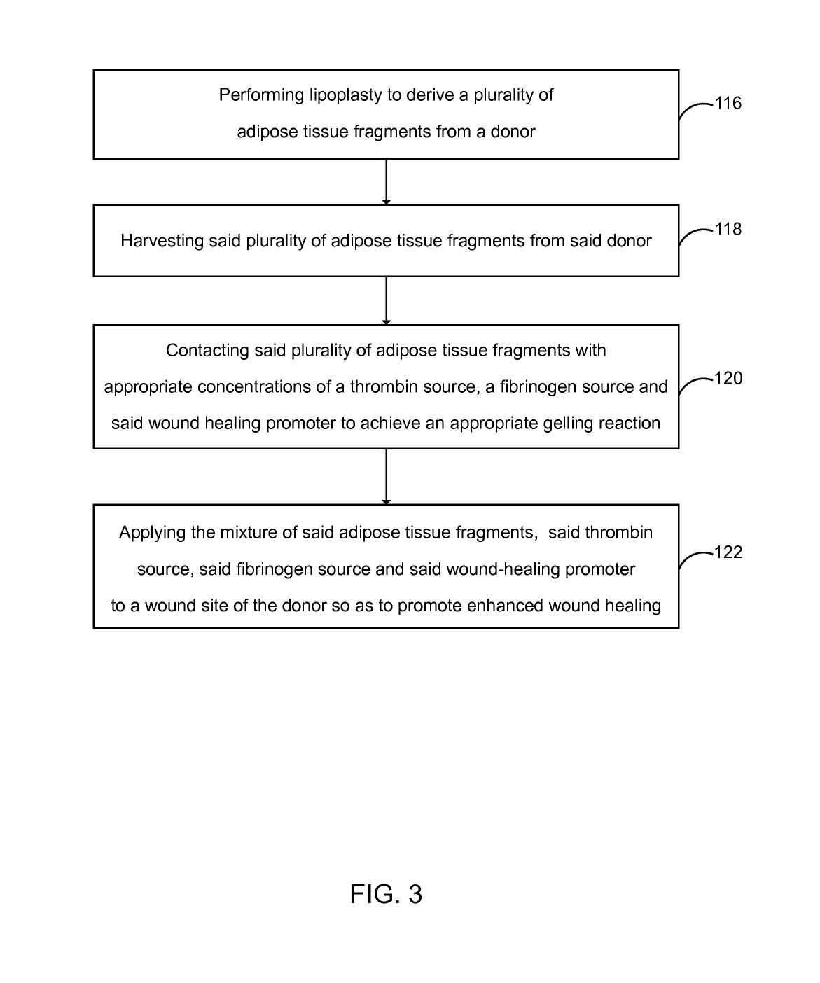

FIG. 3 illustrates an alternative operational flow chart of a method for preparing an improved adipose tissue biocomposite graft with a wound-healing promoter to serve a wide range of medical applications in accordance with another aspect the present invention. In this preferred method, lipoplasty is performed to derive a plurality of adipose tissue fragments from a donor, as shown in block 116. Said plurality of adipose tissue fragments are harvested from said donor as indicated at block 118. In next step, as shown in block 120, said plurality of adipose tissue fragments are contacted with appropriate concentrations of a thrombin source, a fibrinogen source and a wound-healing promoter to achieve an appropriate gelling reaction. Finally, as shown in block 122, the mixture of said adipose tissue fragments, said thrombin source, said fibrinogen source and said wound-healing promoter are applied to a wound site of the donor so as to promote wound healing.

While practicing the above-disclosed method, said wound-healing promoter is employed to enhance wound healing process. Said wound-healing promoter is selected from a group consisting of: platelet rich plasma, mesenchymal stem cells and nucleated blood cells. Said promotion of wound-healing includes at least one of the effects of: promoting hemostasis, reducing time for wound closure, reducing post-surgical wound complications, and reducing scarring. In a preferred embodiment, said adipose tissue biocomposite graft prior to gelling reaction is supplemented with a biologically active agent selected from a group consisting of: cytokines, hormones, drugs including germicides, antibiotics, analgesics, local anesthetic agents, biological response modifiers, bone chips, synthetic bone graft materials, collagen and extracellular matrix.

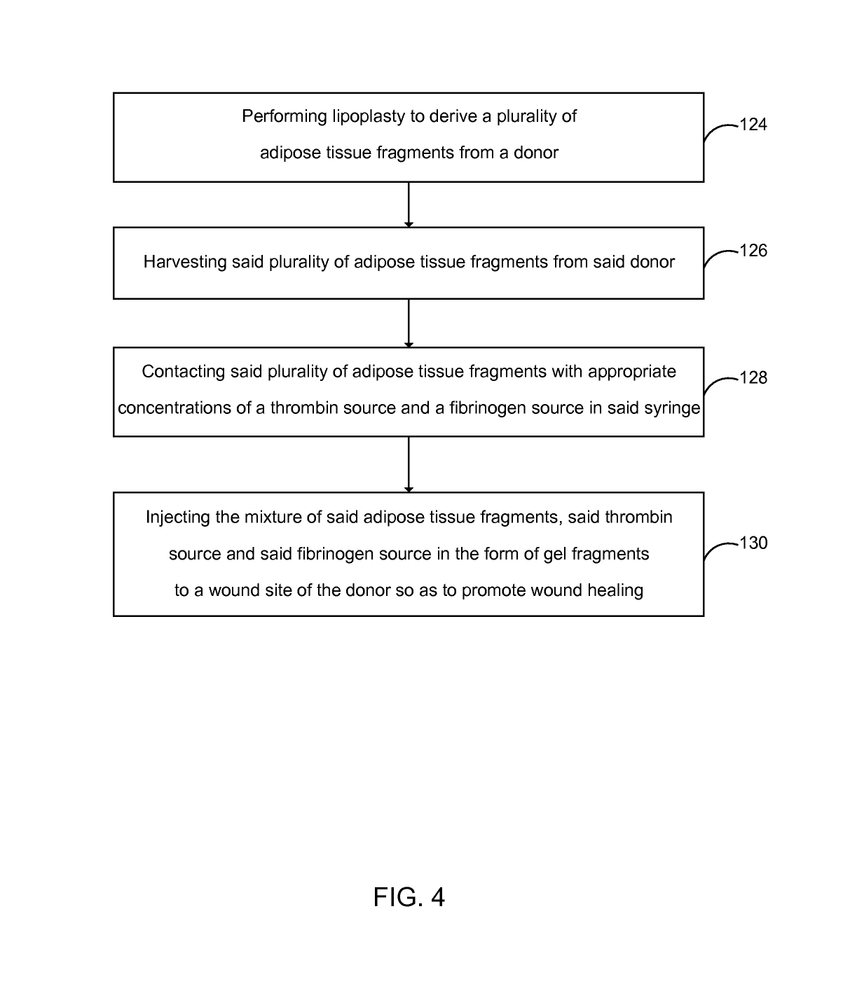

Referring to FIG. 4, an alternative operational flow chart of a method for preparing an improved adipose tissue biocomposite graft utilizing a syringe to serve a wide range of medical applications in accordance with another aspect of the present invention is illustrated. In this method, lipoplasty is performed to derive a plurality of adipose tissue fragments from a donor, as shown in block 124. Said plurality of adipose tissue fragments are harvested from said donor as indicated at block 126. Said plurality of adipose tissue fragments contains at least one viable stem cell. Next, said plurality of adipose tissue fragments are contacted with appropriate concentrations of a thrombin source and a fibrinogen source in said syringe as shown in block 128. Finally, as indicated at block 130, the mixture of said adipose tissue fragments, said thrombin source and said fibrinogen source are injected in the form of gel fragments to a wound site of the donor so as to promote wound healing.

With reference to the above-discussed method, the reaction constituents of tissue fragments, thrombin, and fibrinogen are mixed in liquid form to fill a cavity in or on the body. This type of molding is known as in situ molding. In this instant case, the wound site in the body serves as an in situ mold where the gelling reaction occurs and the tissue biocomposite graft takes the structure of the wound site. The wound site can be a normal anatomical structure, a cavity present as part of pathology or birth defect or a cavity formed by trauma, injury or wound to the body. Alternatively, the wound site can be formed by the injection of the mixture into the body through a cannula. When in situ molding is performed, the tissue biocomposite graft has the potential to serve as a sealant in which oozing bleeding can be arrested. Further, the applicant's biocomposite structure control may be achieved in situ by topical applications. Such topical applications include spraying, painting, pouring, spreading or injecting the biocomposite into the body. In this case, the mixture of tissue fragments, thrombin source and fibrinogen source are delivered prior to its gelling reaction so that the mixture is still in its liquid phase when administered. After delivery, the gelling reaction progresses and the biocomposite graft becomes a solid or semi-solid in the three dimensional structure of the body site it occupies.

In the present invention, the biocomposite graft is prepared by harvesting disassociated tissue fragments from the body and then reconstructing the tissue fragments into molded three-dimensional structures in which a portion of the volume of the graft is living tissue. Specifically, the adipose tissue fragments constitute 10% to 90% of the total volume of said adipose tissue biocomposite graft. More preferably, the fraction of the tissue fragments comprising the greatest collective volume of the biocomposite have a median diameter greater than 100 microns and less than 5000 microns and these same tissue fragments have a median cell number of greater than 10² cells per fragment but less than 10¹⁰ cells per fragment and 70% or more of the cells contained in these same tissue fragments are viable at the time of application to the body. The tissue biocomposite graft may be prepared and used most desirably within the one surgical procedure and within the operation theatre. This simplicity of sourcing and processing the living tissue within a single surgical procedure enables significant savings in costs to the healthcare system. The adipose tissue graft comprising living cells is particularly intended to promote wound repair by reducing the risk of post-surgical complications.

The adipose tissue biocomposite graft of the present invention is a heterogeneous composition of biological materials including viable cells in the form of adipose tissue fragments that may be supplemented with bioactive agents such as cytokines, growth factors, biological response modifiers and drugs to enhance the therapeutic potential of the material. In addition, the adipose tissue biocomposite graft may be treated with an anticoagulant to prevent the premature conversion of fibrinogen to fibrin by inhibiting the formation of thrombin in the fibrinogen source. For the present invention, the preferred anticoagulant contains at least one chemical selected from the group consisting of sodium citrate, potassium citrate, lithium citrate, and EDTA. The use of heparin and anticoagulants that result in direct inactivation of thrombin by working in concert with anti-thrombin III should be avoided in the biocomposite composition.

In the present invention, preferred method of generating tissue fragments from the donor's body is designated as lipoplasty. Briefly, lipoplasty utilizes a cannula, suction source and a harvest chamber to harvest the generated tissue fragments. Lipoplasty is carried out in such a way that the substantial majority of the cells in adipose tissue fragments remain viable. The adipose tissue fragments are derived using lipoplasty methods selected from a group consisting of: suction assisted lipoplasty (SAL), ultra-sound assisted lipoplasty (USAL), power assisted lipoplasty (PAL), syringe assisted lipoplasty (SAL), laser assisted lipoplasty (LAL) and water jet assisted lipoplasty (WJAL).

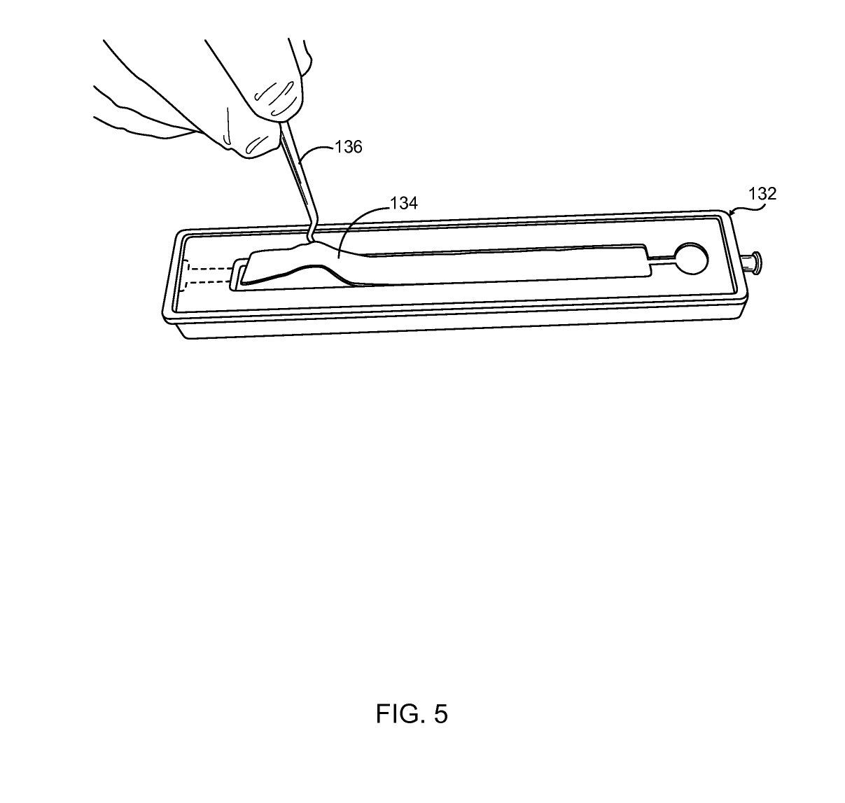

FIG. 5 illustrates the adipose tissue biocomposite graft 134 prepared using a rectangular three dimensional mold cavity 132 in accordance with the exemplary embodiment of the present invention. The adipose tissue biocomposite graft 134 is prepared by injecting the mixture of the adipose tissue fragments, said thrombin source and said fibrinogen source into the rectangular three dimensional mold cavity 132 to achieve the gelling reaction and to confer a three dimensional shape to said adipose tissue biocomposite graft. As shown, the adipose tissue biocomposite graft is removed from the three dimensional mold cavity 132 by an operator using a hand tool 136. The hand tool 136 is selected from a group consisting of: forceps and a pair of tweezers. After molding, the adipose tissue biocomposite graft 134 retains the rectangular shape of the mold cavity 132. The molded strip of the adipose tissue biocomposite graft 134 can be used by a surgeon to reduce post surgical complications.

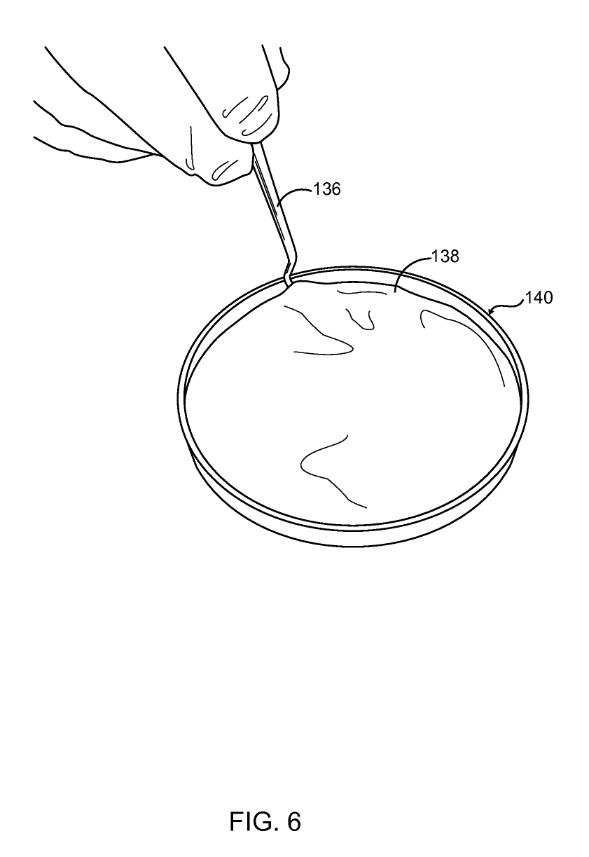

FIG. 6 illustrates the adipose tissue biocomposite graft 138 prepared using a circular three dimensional mold cavity 140 in accordance with the exemplary embodiment of the present invention. The adipose tissue biocomposite graft 138 is removed from the circular mold cavity 140 by the operator using the hand tool 136. The hand tool 136 is used around the edges of the circular mold 140 to release the graft 138 from the circular mold 140. In this case, the adipose tissue biocomposite graft 138 retains the three dimensional shape of the circular mold cavity 140. The molded biocomposite 138 thus formed possess high tensile strength and elasticity, which allows it to be easily moved and handled by the operator without breaking or tearing.

The method of preparing molded adipose tissue biocomposite employing the above-specified three dimensional molds is known as ex vivo molding. The three dimensional mold used in this type of molding is of a size, shape and dimension necessary to control structure of said adipose tissue biocomposite graft. The substantially stable three-dimensional shape is derived by the delivery of the biocomposite elements in a liquid state to the mold cavity. After introduction to the mold, the gelling reaction occurs by the chemistry of the reactants, catalysts and substrates contained in the tissue fragment-suspending medium. The gel thus formed retains the three dimensional shape of the mold when carefully removed from the mold against surface tension forces and gravity for a substantial period of time. The molded biocomposite graft demonstrates elasticity and can be sutured or held in place by it acting as a sealant. Therefore, the adipose tissue graft material can be obtained in any desired size, shape, or dimension by selecting the appropriate mold apparatus.

All of the above discussed methods and embodiments offer the advantage of preparing a new and valuable tool for the presentation of tissue biocomposite grafts to the body to achieve standardized and more reliable tissue grafting method. By employing the present invention, specific molds can be customized by surgeons to carry out defined surgical procedures that will reduce the time and increase reproducibility and reliability of the desired medical applications. The method and materials described for producing the disclosed tissue biocomposite graft preparation are rapid, reliable and easy to use such that they may be used in a variety of different circumstances for performing biological, pharmacologic or toxicology studies. The methods discussed herein are sufficiently simple to perform that users of varying capabilities of know-how posses the required skills and knowledge to prepare the graft. Further, the present method minimizes the amount of hands-on time and total time for grafting procedure. The present invention also provides a means for a single mold device to provide multiple castings of tissue biocomposite grafts for an individual donor.

All of the above-mentioned embodiments of the adipose tissue biocomposite graft and variations thereon may be used for plastic surgery, urology, neurosurgery, orthopedics, dentistry and a wide variety of other medical applications. Such medical applications may include cosmetic, therapeutic and surgical procedures. The autologous adipose graft system represents the safest and potentially the most cost effective way to treat wounds and may be used for civilian and military wounds, trauma, burn and reconstruction applications in remote locations. The adipose biocomposite graft is characterized by good handling properties such as high elasticity and high tensile strength. In the present invention, the tissue fragments can dominate the relative volume of the tissue biocomposite graft with only a minor amount of the volume constituting the extra-cellular gel. For these reasons, the adipose tissue biocomposite can provide an abundant amount of autologous therapeutic cells to be used to treat chronic wounds to enhance the body's natural healing process.

It is possible through careful and customized methods to create adipose tissue biocomposite graft that perform beyond those in the prior art, and may be prepared more simply and potentially the most cost effective than those in the prior art. Using methods described herein, clinically useful biocomposite graft is created at the point of care that is composed substantially of living cells that can be readily handled due to its suitable tensile strength and elasticity.

The structure of the biocomposite graft is dependent on the volume of the adipose tissue fragments and concentrations of said fibrinogen source and said thrombin source, and can be practiced in orchestration with several wound-healing promoters to promote wound-healing process. Promotion of wound healing includes at least one of the effects of: promoting hemostasis, reducing time for wound closure, reducing post-surgical wound complications, reduced scarring and achieving a desirable cosmetic effect.

It is an object to provide a tissue biocomposite graft that can be supplemented with supplements such as a single cell suspension, drug or other graft-modifying agent. These supplements include stem cell concentrates, platelet rich plasma, cytokines, growth factors, antibiotics, analgesics, and other drugs. By supplementing the graft with such materials, customized and more therapeutic grafts can be prepared. In this particular context, the supplements should be added prior to the initiation of the gelling reaction.

Preparation of an adipose biocomposite graft for wound healing can be performed in the following non-limiting exemplary manner. In preparation, three 10 ml syringes designated A, B and C and a three-way stopcock are prepared as described below. Examples of molds used for casting the adipose biocomposite are also provided below.

In this exemplary method, a syringe A is filled with 5 to 8 ml of adipose tissue fragments obtained from a suction canister containing lipoaspirate following lipoplasty. A preferred cannula for harvesting the adipose tissue from the body has an opening of 3 to 5 mm. A preferred lipoplasty system is manufactured by MicroAire Aesthetics of Charlottesville, Va. The syringe is preferably stored in an upright position with its plunger in the top position for approximately 10 to 30 minutes such that the lighter adipose tissue fragments and excess tumescent fluid become separated due to differences in density. The excess tumescent fluid is removed from the syringe by pressing down the plunger forcing the aqueous tumescent fluid to leave the syringe while retaining 4 ml of adipose tissue fragments.

A syringe B is filled with 4 ml volume of normal human plasma having a fibrinogen concentration of 2-6 mg/mL. One may derive the plasma by centrifuging whole blood anticoagulated with a calcium chelating agent such as citrate. For example, centrifugation of a vacutainer containing sodium citrate as the anticoagulant and 10 ml of whole blood for 2,000 G for 10 minutes is sufficient to cause separation of the blood elements from the plasma. The plasma may be selectively removed from the vacutainer using a needle and syringe.

A syringe C is filled with 1 ml of thrombin solution containing 100 U/mL of bovine thrombin. A source of medical grade thrombin is distributed by King Pharmaceuticals of Bristol, Tenn. designated as Thrombin, Topical (BOVINE ORIGIN), U.S.P., with the trade name THROMBIN-JMI. A vial containing 5,000 international units of bovine thrombin is preferably first reconstituted with 5 ml of saline diluent to create a 1,000 U/ml solution. This solution is further diluted 10 fold by adding 1 ml of 1,000 U/mL thrombin to 9 ml of saline to create a 100 U/mL solution. One ml of this solution is aspirated into a 10 ml syringe.

Next in this exemplary method, a standard three-way stopcock may be selected for allowing the mixing the contents of the three syringes. For example, a suitable medical grade stopcock is sold by Qosina from Edgewood, N.Y. with the part number 13813 and that includes 2 female luer locks and 1 male luer lock.

If it is desired to use tubing as the mold for the adipose biocomposite, i.e., spaghetti shape, an extension tubing such as Qosina part number 33061 may be utilized which is 20 inches in length and has internal diameter of 0.094 inches.

If it is desired to make a circular adipose biocomposite, i.e., pancake shaped, a petri dish such as those sold by Sigma Aldrich, St. Louis including Corning CLS3295 culture dishes having a depth of 60 mm and height of 15 mm.

Once the three syringes are prepared, in this exemplary method the adipose tissue fragment syringe A is attached to the stopcock to one luer lock port and the plasma syringe B is attached to a second luer lock port on the stopcock. The contents of the syringes A and B are then intermingled by passing the full contents of the syringes in and out several times between the two syringes until thoroughly mixed by alternatively depressing the plunger of the two syringes with the stopcock handle turned to allow connection between the two syringes. For example, 3 to 6 times of passing the fluids between the two syringes is sufficient to achieve good intermingling of the two fluids. After mixing, the combined fluids are fully delivered into Syringe A and the empty syringe B is removed from the stopcock. The thrombin containing syringe C is then attached to the stopcock. The process of mixing the thrombin fluid with the plasma and adipose fragment mixture is repeated by passaging of the fluids between Syringes A and C. For example, 3 times of passing the fluid is sufficient to achieve mixing. After mixing, the fluid contents are fully loaded into Syringe A. With these concentrations of thrombin and fibrinogen, gelling reaction will occur in approximately 15 to 30 seconds.

If it is desired to cast a mold, the contents of the syringe A now containing thrombin, fibrinogen, adipose tissue fragments may be passed into the mold within 10 seconds of mixing. The mold is preferably left undisturbed for a period of at least one minute to allow the gelling reaction to occur undisturbed. After gelling has occurred, the molded adipose composite may then be removed using forceps. If tubing is used as the mold, the adipose composite may be removed from the tubing by flushing with saline solution dispelling the composite out of the tubing.

If it is desired to deliver the adipose biocomposite as gel fractions, the gelling reaction may be allowed to occur in the syringe containing the mixture of thrombin, fibrinogen and adipose tissue fragments. The gel fragments can be readily ejected from the syringe by depressing the plunger at the desired tissue site to treat a wound.

Each of the examples above confers improved handling properties of adipose tissue fragments which are otherwise amorphous fluids.

The foregoing description of the preferred embodiment of the present invention has been presented for the purpose of illustration and description. It is not intended to be exhaustive or to limit the invention to the precise form disclosed. Many modifications and variations are possible in light of the above teachings. For instance, allogeneic and xenogeneic tissue fragments can be utilized in the practice of the present invention. Further, standard dissection methods may be integrated into the manufacture process to create adipose tissue fragments. Further, adding thrombin or fibrinogen source to the adipose tissue fragments can be done in either order with good results of providing an adipose biocomposite graft containing viable cells suitable for treatment of wounds being achieved. It is intended that the scope of the present invention not be limited by this detailed description, but by the claims and the equivalents to the claims appended hereto.

I claim:

1. A method for preparing an improved adipose tissue biocomposite graft to serve a wide range of medical applications, the method comprising the steps of:
a) performing lipoplasty to derive a plurality of adipose tissue fragments from a donor;
b) harvesting said plurality of adipose tissue fragments from said donor, said adipose tissue fragments containing at least one viable stem cell;
c) contacting said plurality of adipose tissue fragments with appropriate concentration of thrombin source; and
d) applying the mixture of said adipose tissue fragments and said thrombin source to a wound site of the donor so as to promote wound healing.

2. The method of claim 1 wherein said plurality of adipose tissue fragments comprises autologous adipose tissue fragments contained in lipoaspirate.

3. The method of claim 1 wherein said adipose tissue fragments derive a fibrinogen source from the wound site of the donor.

4. The method of claim 1 wherein said promotion of wound healing includes at least one of the effects of: promoting hemostasis, reducing time for wound closure, reducing post-surgical wound complications, and reducing scarring.

5. The method of claim 1 wherein said adipose tissue fragments are derived using lipoplasty methods selected from a group consisting of: suction assisted lipoplasty (SAL), ultra-sound assisted lipoplasty (USAL), power assisted lipoplasty (PAL), syringe assisted lipoplasty (SAL), laser assisted lipoplasty (LAL) and water jet assisted lipoplasty (WJAL).

6. The method of claim 1 wherein said adipose tissue fragments provides a scaffolding that allows cells from surrounding body site to migrate in and produce new tissue in the donor's body.

7. The method of claim 1 wherein said thrombin source is selected from a group consisting of: autologous thrombin serum, autologous thrombin serum supplemented with ethanol, allogeneic thrombin serum, allogeneic serum supplemented with ethanol, bovine thrombin, recombinant thrombin, and human thrombin derived from pooled plasma.

8. The method of claim 1 wherein said concentration of thrombin source is 0.5 to 500 units/gram of said adipose tissue biocomposite graft.

9. The method of claim 1 wherein said adipose tissue fragments constitute 10% to 90% of the total volume of said adipose tissue biocomposite graft.

10. The method of claim 1 wherein said medical application is selected from a group consisting of: cosmetic, therapeutic and surgical procedures.

11. A method for preparing an improved adipose tissue biocomposite graft to serve a wide range of medical applications, the method comprising the steps of:
a) performing lipoplasty to derive a plurality of adipose tissue fragments from a donor;
b) harvesting said plurality of adipose tissue fragments from said donor, said adipose tissue fragments containing at least one viable stem cell;
c) contacting said plurality of adipose tissue fragments with appropriate concentrations of a thrombin source and a fibrinogen source to achieve an appropriate gelling reaction; and
d) applying the mixture of said adipose tissue fragments, said thrombin source and said fibrinogen source to a wound site of the donor so as to promote wound healing.

12. The method of claim 11 wherein step (d) further comprises: applying said mixture of said adipose tissue fragments, said thrombin source and said fibrinogen source to the wound site in the form of: a liquid biocomposite, a molded gel biocomposite and gel biocomposite fragments.

13. The method of claim 12 wherein said liquid biocomposite is prepared by mixing said adipose tissue fragments, said thrombin source, and said fibrinogen source in liquid form to conform to said wound site in the donor's body.

14. The method of claim 12 wherein said molded gel biocomposite is prepared by:
a) injecting the mixture of said adipose tissue fragments, said thrombin source and said fibrinogen source into a three dimensional mold cavity to achieve the gelling reaction and to confer a three dimensional shape to said adipose tissue biocomposite graft;
b) removing said adipose tissue biocomposite graft from said three dimensional mold cavity; and
c) applying said adipose tissue biocomposite graft to a wound site of the donor so as to promote wound healing.

15. The method of claim 14 wherein said three dimensional mold cavity is of a size, shape and dimension to control the structure of said adipose tissue biocomposite graft.

16. The method of claim 11 wherein said promotion of wound healing includes at least one of the effects of: promoting hemostasis, reducing time for wound closure, reducing post-surgical wound complications, and reducing scarring.

17. The method of claim 11 wherein said adipose tissue fragments are derived using lipoplasty methods selected from a group consisting of: suction assisted lipoplasty (SAL), ultra-sound assisted lipoplasty (USAL), power assisted lipoplasty (PAL), syringe assisted lipoplasty (SAL), laser assisted lipoplasty (LAL) and water jet assisted lipoplasty (WJAL).

18. The method of claim 11 wherein said thrombin source is selected from a group consisting of: autologous thrombin serum, autologous thrombin serum supplemented with ethanol, allogeneic thrombin serum, allogeneic thrombin serum supplemented with ethanol, bovine thrombin, recombinant thrombin, and human thrombin derived from pooled plasma.

19. The method of claim 11 wherein said fibrinogen source is selected from a group consisting of: autologous whole blood anti-coagulated with a calcium-chelating agent, plasma anti-coagulated with a calcium-chelating agent, platelet rich plasma with its associated growth factors, autologous plasma, autologous platelet rich plasma, plasma and collagen mixture, purified allogeneic fibrinogen and other naturally occurring adhesive glycoproteins to promote adhesion to collagen.

20. The method of claim 11 further comprising controlling the structure of said adipose tissue biocomposite graft by controlling the relative percentage of the graft volume derived from adipose tissue fragments and controlling concentrations of said fibrinogen source and said thrombin source.

21. The method of claim 11 wherein said concentration of thrombin source is 0.5 to 500 units/gram of said adipose tissue biocomposite graft.

22. The method of claim 11 wherein said concentration of fibrinogen source is 0.1 to 60 mg/gram of said adipose tissue biocomposite graft.

23. The method of claim 11 wherein said adipose tissue fragments constitute 10% to 90% of the total volume of said adipose tissue biocomposite graft.

24. The method of claim 11 wherein said medical application is selected from a group consisting of: cosmetic, therapeutic and surgical procedures.

25. A method for preparing an improved adipose tissue biocomposite graft with a wound-healing promoter to serve a wide range of medical applications, the method comprising the steps of:
a) performing lipoplasty to derive a plurality of adipose tissue fragments from a donor;
b) harvesting said plurality of adipose tissue fragments from said donor, said adipose tissue fragments containing at least one viable stem cell;
c) contacting said plurality of adipose tissue fragments with appropriate concentrations of a thrombin source, a fibrinogen source and said wound healing promoter to achieve an appropriate gelling reaction; and
d) applying the mixture of said adipose tissue fragments, said thrombin source, said fibrinogen source and said wound-healing promoter to a wound site of the donor so as to promote enhanced wound healing.

26. The method of claim 25 wherein said wound-healing promoter is selected from a group consisting of: platelet rich plasma, mesenchymal stem cells and nucleated blood cells.

27. The method of claim 25 wherein step (d) further comprises: applying said mixture of said adipose tissue fragments, said thrombin source and said fibrinogen source to the wound site in the form of: a liquid biocomposite, a molded gel biocomposite and gel biocomposite fragments.

28. The method of claim 27 wherein said liquid biocomposite is prepared by mixing said adipose tissue fragments, said thrombin source and said fibrinogen source in liquid form to conform to said wound site in donor's body.

29. The method of claim 27 wherein preparation of said molded gel biocomposite comprises:
a) injecting the mixture of said adipose tissue fragments, said thrombin source and said fibrinogen source into a three dimensional mold cavity to achieve the gelling reaction and to confer a three dimensional shape to said adipose tissue biocomposite graft;
b) removing said adipose tissue biocomposite graft from said three dimensional mold cavity; and
c) applying said adipose tissue biocomposite graft to a wound site of the donor so as to promote wound healing.

30. The method of claim 29 wherein said three dimensional mold cavity is of a size, shape and dimension to control the structure of said adipose tissue biocomposite graft.

31. The method of claim 25 wherein said promotion of wound healing includes at least one of the effects of: promoting hemostasis, reducing time for wound closure, reducing post-surgical wound complications, and reducing scarring.

32. The method of claim 25 wherein said adipose tissue fragments are derived using lipoplasty methods selected from a group consisting of: suction assisted lipoplasty (SAL), ultra-sound assisted lipoplasty (USAL), power assisted lipoplasty (PAL), syringe assisted lipoplasty (SAL), laser assisted lipoplasty (LAL) and water jet assisted lipoplasty (WJAL).

33. The method of claim 25 wherein said thrombin source is selected from a group consisting of: autologous thrombin serum, autologous thrombin serum supplemented with ethanol, allogeneic thrombin serum, allogeneic serum supplemented with ethanol, bovine thrombin, recombinant thrombin, and human thrombin derived from pooled plasma.

34. The method of claim 25 wherein said fibrinogen source is selected from a group consisting of: autologous whole blood anti-coagulated with a calcium-chelating agent, platelet rich plasma with its associated growth factors, autologous plasma, autologous platelet rich plasma, plasma and collagen mixture, purified allogeneic fibrinogen and other naturally occurring adhesive glycoproteins to promote adhesion to collagen.

35. The method of claim 25 wherein said adipose tissue biocomposite graft prior to gelling reaction are supplemented with a biologically active agent selected from a group consisting of: cytokines, hormones, drugs including germicides, antibiotics, analgesics, local anesthetic agents, biological response modifiers, bone chips, synthetic bone graft materials, collagen and extracellular matrix.

36. The method of claim 25 further comprising controlling the structure of said biocomposite graft by controlling the relative percentage of the graft volume derived from adipose tissue fragments and controlling concentrations of said fibrinogen source and said thrombin source.

37. The method of claim 25 wherein said adipose tissue fragments constitute 10% to 90% of the total volume of said adipose tissue biocomposite graft.

38. The method of claim 25 wherein said medical application is selected from a group consisting of: cosmetic, therapeutic and surgical procedures.

39. A method for preparing an improved adipose tissue biocomposite graft utilizing a syringe to serve a wide range of medical applications, the method comprising the steps of:
a) performing lipoplasty to derive a plurality of adipose tissue fragments from a donor;
b) harvesting said plurality of adipose tissue fragments from said donor, said adipose tissue fragments containing at least one viable stem cell;
c) contacting said plurality of adipose tissue fragments with appropriate concentrations of a thrombin source and a fibrinogen source in said syringe; and
d) injecting the mixture of said adipose tissue fragments, said thrombin source and said fibrinogen source in the form of gel fragments to a wound site of the donor so as to promote wound healing.

40. The method of claim 39 wherein said plurality of adipose tissue fragments comprises autologous adipose tissue fragments contained in lipoaspirate.

41. The method of claim 39 wherein the mixture of said plurality of adipose tissue fragments, said thrombin source and said fibrinogen source takes the structure of the wound site and said wound site serves as a mold for the gelling reaction to occur.

42. The method of claim 39 wherein said promotion of wound healing includes at least one of the effects of: promoting hemostasis, reducing time for wound closure, reducing post-surgical wound complications, and reducing scarring.

43. The method of claim 39 wherein said thrombin source is selected from a group of consisting of: autologous thrombin serum, autologous thrombin serum supplemented with ethanol, allogeneic thrombin serum, allogeneic serum supplemented with ethanol, bovine thrombin, recombinant thrombin, and human thrombin derived from pooled plasma.

44. The method of claim 39 wherein said fibrinogen source is selected from a group consisting of: autologous whole blood anti-coagulated with a calcium-chelating agent, platelet rich plasma with its associated growth factors, autologous plasma, autologous platelet rich plasma, plasma and collagen mixture, purified allogeneic fibrinogen and other naturally occurring adhesive glycoproteins to promote adhesion to collagen.

45. The method of claim 39 wherein said adipose tissue fragments constitute 10% to 90% of the total volume of said adipose tissue biocomposite graft.
